(19) United States
(12) Patent Application Publication
(10) Pub. No.: US 2009/0093491 A1
(43) Pub. Date: Apr. 9, 2009

(54) Diazine Azole Derivatives, Their Manufacture and Use as Pharmaceutical

(76) Inventors: Wolfgang Jenni, Muenchen (DE); Thomas Von Hirschheydt, Penzberg (DE); Edgar Voss, Bichl (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/990,326

(22) PCT Filed: Sep. 28, 2006
(86) PCT No.: PCT/EP2006/009435

§ 371 Date: Feb. 8, 2008

(30) Foreign Application Priority Data
Sep. 30, 2005 (EP) 05021467.5

(51) Int. Cl. A61K 31/501; C07D 413/14; A61K 31/506; A61K 31/497

(52) U.S. Cl. 514/252.05; 544/238; 544/298; 544/405; 514/255.05; 514/269

(57) ABSTRACT

Objects of the present invention are the compounds of formula I

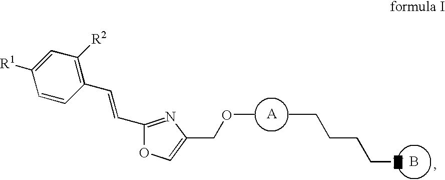

their pharmaceutically acceptable salts, enantiomeric forms, diastereoisomers and racemates, the preparation of the above-mentioned compounds, pharmaceutical compositions containing them and their manufacture, as well as the use of the above-mentioned compounds in the control or prevention of illnesses such as cancer.

The present invention relates to novel diazine azole derivatives, to a process for their manufacture, pharmaceutical compositions containing them and their manufacture as well as the use of these compounds as pharmaceutically active agents.

### BACKGROUND OF THE INVENTION

The treatment of cancer diseases is of great importance in medicine. There is a worldwide need for effective cancer therapies in order to achieve a treatment which is appropriate to a patient and is target-orientated. This can be seen in the large number of scientific studies which have recently appeared in the fields of applied oncology and fundamental research relating to cancer therapy.

The effects of tumor inhibitors are due to a very wide variety of mechanisms, only some of which are known. It is not unusual for known tumor drugs to be found to have new mechanisms of action. This is also to be expected in the case of the compounds according to the invention. Many tumor drugs act by way of mechanisms such as blockading the mechanism of cell division in the cell, preventing the tumor from being supplied with nutrients and oxygen (antiangiogenesis), preventing metastasis, preventing the reception and the onward transmission of growth signals to the tumor cell or forcing the tumor cell into programmed cell death (apoptosis).

Because they have different mechanisms of action, including interacting with different intracellular targets, the clinically relevant cytostatic agents are frequently administered in combination in order to achieve a synergistic therapeutic effect.

WO 98/03505, WO 01/77107, WO 03/031442 and WO 03/059907 relate to heterocyclic compounds as tyrosine kinase inhibitors which are useful as anticancer agents.

### SUMMARY OF THE INVENTION

The present invention relates to compounds of the general formula I,

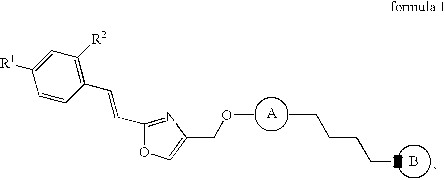

wherein
R¹ is halogenated alkyl, halogenated alkoxy or halogen;
R² is hydrogen or halogen;
ring A is

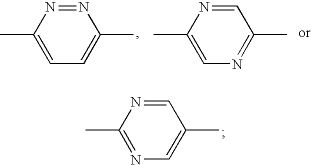

ring B is

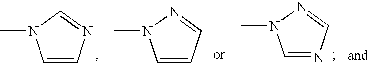

pharmaceutically acceptable salts thereof.

The compounds of the present invention show anti-proliferative activity. Objects of the present invention are the compounds of formula I and their pharmaceutically acceptable salts, enantiomeric forms, diastereoisomers and racemates, the preparation of the above-mentioned compounds, pharmaceutical compositions containing them and their manufacture as well as the use of the above-mentioned compounds in the control or prevention of illnesses, especially of illnesses and disorders as mentioned above like common human cancers (e.g. breast cancer, gastrointestinal cancer (colon, rectal or stomach cancer), leukaemia and ovarian, bronchial and pancreatic cancer) or in the manufacture of corresponding pharmaceutical compositions.

### DETAILED DESCRIPTION OF THE INVENTION

As used herein, the term “alkyl” means a saturated, straight-chain or branched-chain hydrocarbon containing from 1 to 5, preferably 1 to 3, carbon atoms, such as methyl, ethyl, n-propyl, isopropyl, n-butyl, 2-butyl, t-butyl, n-pentyl, 3-methyl-butyl or 2-methyl-butyl.

As used herein the term “alkoxy” means an alkyl group as defined above which attached via an oxygen (alkyl-O—).

As used herein, the term “halogenated alkyl” means an alkyl group as defined above which is substituted with one or several halogen atoms, preferably fluorine or chlorine, especially fluorine. Examples are trifluoromethyl, 2,2,2-trifluoroethyl, perfluoroethyl and the like, preferably trifluoromethyl.

The term “halogenated alkoxy” as used herein means an alkoxy group as defined above which is substituted one or several times by halogen, preferably by fluorine or chlorine, especially by fluorine. Examples are difluoromethoxy, trifluoromethoxy, 2,2,2-trifluoroethoxy, perfluoroethoxy and the like, preferably trifluoromethoxy and difluoromethoxy and especially trifluoromethoxy.

The term “halogen” as used herein means fluorine, chlorine and bromine, preferably fluorine or chlorine.

In a preferred embodiment, the term “halogen” as used in the definition of R¹ denotes fluorine or chlorine, preferably chlorine and the term “halogen” as used in the definition of R² denotes fluorine or chlorine, preferably fluorine.

As used herein, in relation to mass spectrometry (MS) the term “ES+” refers to positive electrospray ionization mode and the term “APCI+” refers to positive atmospheric pressure chemical ionization mode.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

An embodiment of the invention are the compounds according to formula I,
wherein
R² is hydrogen.

Another embodiment of the invention are the compounds according to formula I,
wherein
R¹ is halogenated alkyl or halogenated alkoxy.

Another embodiment of the invention are the compounds according to formula I,
wherein
R¹ is halogenated alkyl or halogenated alkoxy; and
R² is hydrogen.

Another embodiment of the invention are the compounds according to formula I,
wherein
R¹ is trifluoromethyl, trifluoromethoxy or chlorine; and
R² is hydrogen or fluorine.

Another embodiment of the invention are the compounds according to formula I,
wherein
R¹ is halogenated alkoxy.

Another embodiment of the invention are the compounds according to formula I,
wherein
R¹ is halogenated alkoxy; and
R² is hydrogen.

Another embodiment of the invention are the compounds according to formula I,
wherein
R¹ is halogenated alkyl.

Another embodiment of the invention are the compounds according to formula I,
wherein
R¹ is halogenated alkyl; and
R² is hydrogen.

Another embodiment of the invention are the compounds according to formula I,
wherein
R¹ is halogen.

Another embodiment of the invention are the compounds according to formula I,
wherein
R¹ is halogen; and
R² is hydrogen.

Another embodiment of the invention are the compounds according to formula I,
wherein
ring A is

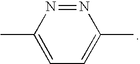

Such compounds, for example, may be selected from the group consisting of:
3-(4-Imidazol-1-yl-butyl)-6-{2-[(E)-2-(4-trifluoromethoxy-phenyl)-vinyl]-oxazol-4-ylmethoxy}-pyridazine;
3-{2-[(E)-2-(4-Chloro-phenyl)-vinyl]-oxazol-4-ylmethoxy}-6-(4-[1,2,4]triazol-1-yl-butyl)-pyridazine;
3-(4-[1,2,4]Triazol-1-yl-butyl)-6-{2-[(E)-2-(4-trifluoromethyl-phenyl)-vinyl]-oxazol-4-ylmethoxy}-pyridazine; and
3-{2-[(E)-2-(2-Fluoro-4-trifluoromethyl-phenyl)-vinyl]-oxazol-4-ylmethoxy}-6-(4-[1,2,4]triazol-1-yl-butyl)-pyridazine.

Another embodiment of the invention are the compounds according to formula I,
wherein
R² is hydrogen; and
ring A is

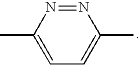

Another embodiment of the invention are the compounds according to formula I,
wherein
ring A is

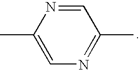

Another embodiment of the invention are the compounds according to formula I,
wherein
R² is hydrogen; and
ring A is

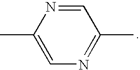

Such compounds, for example, may be selected from the group consisting of:
2-(4-[1,2,4]Triazol-1-yl-butyl)-5-{2-[(E)-2-(4-trifluoromethyl-phenyl)-vinyl]-oxazol-4-ylmethoxy}-pyrazine;
2-{2-[(E)-2-(4-Chloro-phenyl)-vinyl]-oxazol-4-ylmethoxy}-5-(4-[1,2,4]triazol-1-yl-butyl)-pyrazine; and
2-(4-[1,2,4]Triazol-1-yl-butyl)-5-{2-[(E)-2-(4-trifluoromethoxy-phenyl)-vinyl]-oxazol-4-ylmethoxy}-pyrazine.

Another embodiment of the invention are the compounds according to formula I,
wherein
ring A is

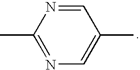

Another embodiment of the invention are the compounds according to formula I,
wherein
R² is hydrogen; and
ring A is

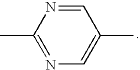

Such compounds, for example, may be selected from the group consisting of:
5-(4-Pyrazol-1-yl-butyl)-2-{2-[(E)-2-(4-trifluoromethoxy-phenyl)-vinyl]-oxazol-4-ylmethoxy}-pyrimidine;
5-(4-Pyrazol-1-yl-butyl)-2-{2-[(E)-2-(4-trifluoromethyl-phenyl)-vinyl]-oxazol-4-ylmethoxy}-pyrimidine; and
2-{2-[(E)-2-(4-Chloro-phenyl)-vinyl]-oxazol-4-ylmethoxy}-5-(4-pyrazol-1-yl-butyl)-pyrimidine.

Another embodiment of the invention are the compounds according to formula I,
wherein
ring B is

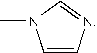

Another embodiment of the invention are the compounds according to formula I,
wherein
ring B is

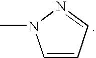

Another embodiment of the invention are the compounds according to formula I,
wherein
ring A is

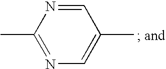

ring B is

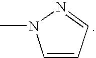

Another embodiment of the invention are the compounds according to formula I,
wherein
R¹ is trifluoromethyl, trifluoromethoxy or chlorine;
R² is hydrogen;
ring A is

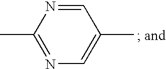

ring B is

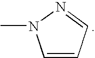

Another embodiment of the invention are the compounds according to formula I,
wherein
ring B is

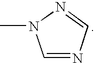

Another embodiment of the invention are the compounds according to formula I,
wherein
R¹ is trifluoromethyl, trifluoromethoxy or chlorine;
R² is hydrogen or fluorine; and
ring B is

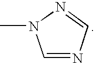

Another embodiment of the invention are the compounds according to formula I,
wherein
R¹ is trifluoromethyl, trifluoromethoxy or chlorine;
R² is hydrogen or fluorine;
ring A is

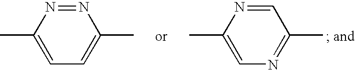

ring B is

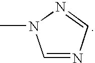

Another embodiment of the invention are the compounds according to formula I,
wherein
R¹ is trifluoromethyl, trifluoromethoxy or chlorine; and
R² is hydrogen or fluorine.
ring A is

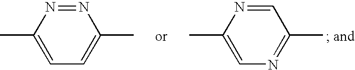

ring B is

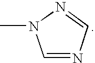

Another embodiment of the invention is a process for the manufacture of the compounds of formula I, wherein
(a) the compound of formula II

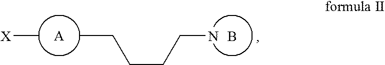

wherein ring A and ring B have the significance as given in formula I above and X is chlorine or bromine,
is reacted with a compound of formula III

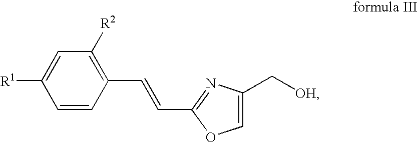

wherein R¹ and R² have the significance given in formula I above, to give the respective compound of formula I;
(c) said compound is isolated from the reaction mixture, and
(d) if desired, converted into a pharmaceutically acceptable salt.

The compounds of formula I, or a pharmaceutically acceptable salt thereof, which are subject of the present invention, may be prepared by any process known to be applicable to the preparation of chemically-related compounds. Such processes, when used to prepare a compound of the formula I, or a pharmaceutically-acceptable salt thereof, are illustrated by the following representative schemes 1 to 2 and examples in which, unless otherwise stated, R¹, R², ring A and ring B have the significance given herein before. Necessary starting materials are either commercially available or they may be obtained by standard procedures of organic chemistry. The preparation of such starting materials is e.g. described within the accompanying examples or in the literature cited below with respect to schemes 1 to 2. Alternatively necessary starting materials are obtainable by analogous procedures to those illustrated which are within the ordinary skill of an organic chemist or they can be prepared according to e.g. U.S. Pat. No. 6,743,924, Goodman, A. J., Tetrahedron 55 (1999) 15067-1507 or Pieterse, K., Chemistry—A European Journal 9 (2003) 5597-5604.

A preferred method for the synthesis of the compounds of formula I is described in scheme 1 in which R¹, R², ring A and ring B have the significance given above and X is chlorine or bromine.

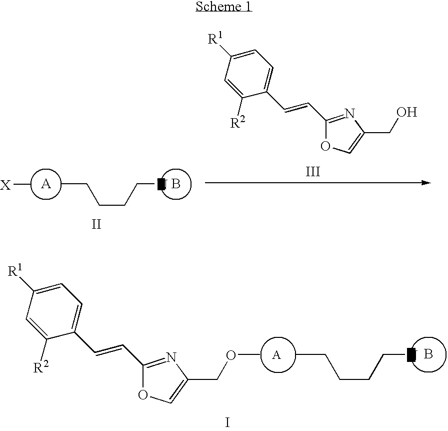

The preparation starts from a halogenated diazine derivative of formula II which is reacted in an addition-elimination reaction with the hydroxymethyl derivatives of formula III. The reaction is typically performed in solvents like tetrahydrofuran (THF), N,N-dimethylformamide (DMF) and mixtures thereof at temperatures from room temperature to 150° C. (heating conditions can vary from oil bath to a microwave reactor), yielding the compounds of formula I. The reaction is carried out in the presence of a non nucleophilic base like sodium tert-butoxide, potassium tert-butoxide, N-ethyl-N,N-diisopropyl amine, triethyl amine or the like.

The hydroxymethyl derivatives of formula III can be obtained from the corresponding chloromethyl derivatives. This reaction is typically performed in a two step procedure, starting with the reaction of corresponding chloromethyl derivatives with sodium or potassium acetate which is typically performed in solvents like N,N-dimethylformamide, N-methylpyrrolidinone, acetonitrile, dimethylsulfoxide and mixtures thereof at temperatures between 50° C. and 140° C. or at reflux. In the second step hydrolysis of the resulting acetates is achieved by standard methods for someone skilled in the art. Typically used bases are e.g. sodium hydroxide (NaOH), potassium hydroxide (KOH) or lithium hydroxide (LiOH) in solvents like water, tetrahydrofuran, N,N-dimethylformamide, dimethylsulfoxide, methanol, ethanol or mixtures thereof at temperatures between 0° C. and 150° C., yielding the hydroxymethyl derivatives of formula III. The preparation of such corresponding chloromethyl derivatives as well as the conversion into the hydroxymethyl derivatives is described e.g. in U.S. Pat. No. 6,743,924.

A preferred method for the synthesis of the halogenated diazine derivatives of formula II is described in scheme 2 in which R¹, R², ring A and ring B have the significance given above, LG is a leaving group such as e.g. iodide, bromide, chloride, p-toluenesulfonate, methanesulfonate, trifluoromethansulfonate and the like, X is chlorine or bromine, Y is bromine or iodine and not both X and Y are bromine.

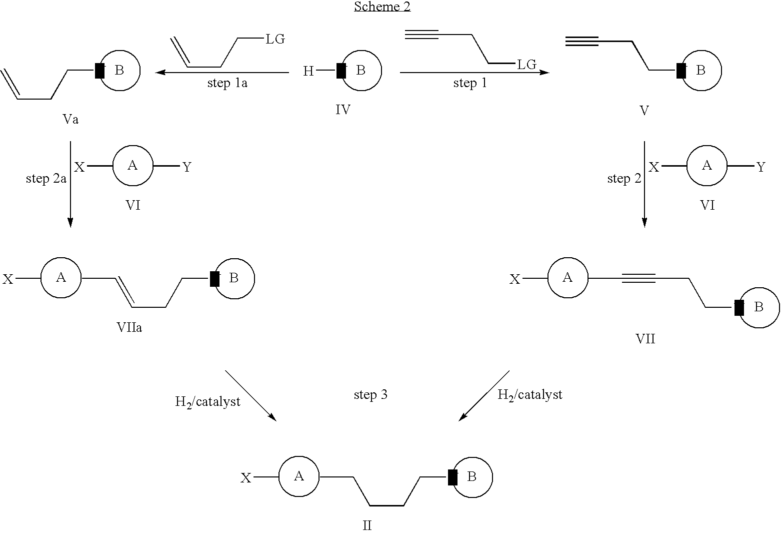

In step 1 an azole of the formula IV is N-alkylated with a suitable but-1-yne derivative, yielding the terminal alkynes of formula V. Typically the N-alkylation is carried out in inert solvents like N,N-dimethylformamide (DMF) or tetrahydrofuran (THF) in the presence of a base like sodium hydride or in alcohols like methanol, ethanol and 2-methylbutan-2-ol in the presence of bases such as sodium methylate or sodium hydroxide and the like. The reaction temperatures may vary from 0° C. to 150° C. Sometimes potassium iodide or sodium iodide is added to the reaction mixture to accelerate the reaction. Suitable leaving groups LG are those typically used in N-alkylation reactions and well known to the skilled artisan. Examples of such leaving groups LG are, among others, the anions of halogens, especially iodide, bromide or chloride, p-toluenesulfonate, methanesulfonate or trifluoromethansulfonate.

In step 2, scheme 2 the dihalodiazines of formula VI are reacted with alkyne derivatives of formula V in a Sonogashira cross-coupling reaction in the presence of catalytic amounts copper iodide and a palladium complex, e.g. Pd(PPh₃)₄, Pd(PPh₃)₂Cl₂ or the like. The reaction is carried out in the presence of a base like triethyl amine, diisopropyl amine, isopropyl amine, piperidine, morpholine or pyrrolidine and in solvents like tetrahydrofurane, N,N-dimetyhylformamide or mixtures thereof at temperatures varying from 20° C. to 120° C. yielding derivatives of formula VII.

The dihalodiazines of formula VI are either commercially available or prepared according to literature protocols, e.g. Goodman, A. J., Tetrahedron 55 (1999) 15067-1507 and Pieterse, K., Chemistry—A European Journal 9 (2003) 5597-5604.

Step 3, scheme 2 is a catalytic hydrogenation which can be carried out using different metal catalysts like palladium, nickel, platinum or platinum dioxide. The catalytically active metals may be supported on typical carriers like activated charcoal, barium sulfate, calcium carbonate or the like. The reaction is typically performed at temperatures between 0° C. and 50° C., at hydrogen pressures between 1 and 4 atm in solvents like methanol, ethanol, tetrahydrofuran, acetone, ethyl acetate and mixtures thereof, yielding the compounds of the formula II.

An alternative route for the preparation of compounds of formula II is also shown in scheme 2 and proceed via the steps 1a, 2a and 3. Starting from the same azoles of formula IV, N-alkylation in step 1a is carried out in a similar manner to step 1 but using but-1-enes to yield the corresponding terminal alkenes of formula Va. These are reacted with the dihalodiazines of formula VI in a Heck cross-coupling reaction in the presence of a palladium complex, e.g. Pd(PPh₃)₄, Pd(PPh₃)₂Cl₂ or the like. The reaction is carried out in the presence of a base like triethyl amine or tributyl amine and in solvents like tetrahydrofuran, N,N-dimetyhylformamide or mixtures thereof at temperatures varying from 20° C. to 120° C. yielding derivatives of formula VIIa. Catalytic hydrogenation as described above gives the compounds of the formula II.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases which are commercially available.

### Pharmacological Activity

The compounds of formula I and their pharmaceutically acceptable salts possess valuable pharmacological properties such as anti-proliferative activity. Consequently the compounds of the present invention are useful in the therapy and/or prevention of proliferative disorders such as cancer. The activity of the present compounds as antiproliferative inhibitors can be demonstrated e.g. by the following biological assay:

### CellTiter-Glo™ Assay in HEK293 Cells

The CellTiter-Glo™ Luminescent Cell Viability Assay (Promega) is a homogeneous method of determining the number of viable cells in culture based on quantitation of the ATP present, which signals the presence of metabolically active cells.

HEK293 cells (human embryonic kidney cell line transformed by Adenovirus 5 fragments, ATCC-No. CRL 1573) are cultivated in Dulbecco's Modified Eagle Medium (DMEM) with Glutamax™ (Invitrogen, 31966-021), 5% Fetal Calf Serum (FCS, Sigma Cat-No. F4135 (FBS)), 100 Units/ml penicillin/100 μg/ml streptomycin (=Pen/Strep from Invitrogen Cat. No. 15140). For the assay the cells are seeded in 384 well plates, 5000 cells per well, in the same medium. The next day the test compounds are added in various concentrations ranging from 3 μM to 0.00015 μM (10 concentrations, 1:3 diluted). After 7 days the CellTiter-Glo™ assay is done according to the instructions of the manufacturer (CellTiter-Glo™ Luminescent Cell Viability Assay, from Promega). In brief: the cell-plate is equilibrated to room temperature for approximately 30 minutes and than the CellTiter-Glo™ reagent is added. The contents are carefully mixed for 15 minutes to induce cell lysis. After 45 minutes the luminescent signal is measured in Victor 2, (scanning multiwell spectrophotometer, Wallac).

### Details:

1. day:
Medium: Dulbecco's Modified Eagle Medium (DMEM) with Glutamax™ (Invitrogen, 31966-021), 5% Fetal Calf Serum (FCS, Sigma Cat-No. F4135 (FBS)), Pen/Strep (Invitrogen Cat. No. 15140).
HEK293 (ATCC-No. CRL 1573): 5000 cells in 60 μl per well of 384 well plate (Greiner 781098, white plates)
Incubate 24 h at 37° C., 5% CO₂
   2. day: Induction (Substance testing):

In general the dilution steeps are 1:3
a) Add 8 μl of 10 mM stock solution of compound to 72 μl DMSO
b) dilute 9× 1:3 (always 30 μl to 60 μl DMSO) in this DMSO dilution row (results in 10 wells with concentrations from 1000 μM to 0.06 μM)
c) dilute each concentration 1:4.8 (10 μl compound dilution to 38 μl medium)
d) dilute each concentration 1:10 (10 μl compound dilution to 90 μl medium)
e) add 10 μl of every concentration to 60 μl medium in the cell plate
   resulting in final concentration of DMSO: 0.3% in every well
   and resulting in final concentration of compounds from 3 μM to 0.00015 μM
   Incubate 168 h (7 days) at 37° C., 5% CO₂

### Analysis:

Add 30 μl CellTiter-Glo™ Reagent/well,
shake 15 minutes at room temperature
incubate further 45 minutes at room temperature without shaking.

### Measurement:

Victor 2 scanning multiwell spectrophotometer (Wallac), Luminescence mode
Determine IC50 with XL-fit (XLfit software (ID Business Solution Ltd., Guilford, Surrey, UK)).

A significant inhibition of HEK293 cell viability was detected, which is exemplified by the compounds shown in Table 1.

TABLE 1
Results:

| Examples | IC50 HEK293 [μM] |
|---|---|
| 3 | 0.93 |
| 6 | 1.34 |
| 1, 2, 4, 5, 8, 10 | 0.2-2.0 |

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Examples of acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g., Stahl, P. H., and Wermuth, G., (editors), Handbook of Pharmaceutical Salts, Verlag Helvetica Chimica Acta (VHCA), Züirich, (2002) or Bastin, R. J., et al., Organic Proc. Res. Dev. 4 (2000) 427-435.

Preferred are the pharmaceutically acceptable salts, which are formed with p-toluenesulfonic acid, naphthalenesulfonic acid, naphthalenedisulfonic acid, methanesulfonic acid and hydrochloric acid.

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

Medicaments or pharmaceutical compositions containing a compound of the present invention or a pharmaceutically acceptable salt thereof and a therapeutically acceptable carrier are also an object of the present invention, as is a process for their production, which comprises bringing one or more compounds of the present invention and/or pharmaceutically acceptable salts and, if desired, one or more other therapeutically valuable substances into a galenical administration form together with one or more therapeutically acceptable carriers.

In accordance with the invention the compounds of the present invention as well as their pharmaceutically acceptable salts are useful in the control or prevention of illnesses. Based on their HER-signalling pathway inhibition and their antiproliferative activity, said compounds are useful for the treatment of diseases such as cancer in humans or animals and for the production of corresponding pharmaceutical compositions. The dosage depends on various factors such as manner of administration, species, age and/or individual state of health.

Another embodiment of the invention is pharmaceutical composition, containing one or more compounds of formula I together with pharmaceutically acceptable carriers.

Still another embodiment of the invention is said pharmaceutical composition for the inhibition of tumor growth.

Still another embodiment of the invention is the use of a compound of formula I for the inhibition of tumor growth.

Still another embodiment of the invention is the use of a compound of formula I for the treatment of cancer.

Still another embodiment of the invention is the use of a compound of formula I for the manufacture of corresponding pharmaceutical compositions for the inhibition of tumor growth.

Another embodiment of the invention is a pharmaceutical composition comprising a therapeutically effective amount of a compound according to formula I as active ingredients and a pharmaceutically acceptable carrier.

Another embodiment of the invention is a method of treating cancer comprising administering to a person in need thereof a therapeutically effective amount of a compound according to formula I.

Another embodiment of the invention is a method of treating colorectal cancer, breast cancer, lung cancer, prostate cancer, pancreatic cancer, gastric cancer, bladder cancer, ovarian cancer, melanoma, neuroblastoma, cervical cancer, kidney cancer or renal cancer, leukemias or lymphomas comprising administering to a person in need thereof a therapeutically effective amount of a compound according to formula I.

The above-mentioned pharmaceutical compositions can be obtained by processing the compounds according to this invention with pharmaceutically acceptable, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

### Pharmaceutical Compositions Comprise e.g. the Following:

### a) Tablet Formulation (Wet Granulation):

| Item | Ingredients | mg/tablet | | | |
|---|---|---|---|---|---|
| 1. | Compound of formula (I) | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure:

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### b) Capsule Formulation:

| Item | Ingredients | mg/capsule | | | |
|---|---|---|---|---|---|
| 1. | Compound of formula (I) | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure:

1. Mix items 1, 2 and 3 in a suitable mixer for 30 minutes.
2. Add items 4 and 5 and mix for 3 minutes.
3. Fill into a suitable capsule.

The following examples and references are provided to aid the understanding of the present invention, the true scope of which is set forth in the appended claims. It is understood that modifications can be made in the procedures set forth without departing from the spirit of the invention.

### EXAMPLE 1

### 3-(4-Imidazol-1-yl-butyl)-6-{2-[(E)-2-(4-trifluoromethoxy-phenyl)-vinyl]-oxazol-4-ylmethoxy}-pyridazine

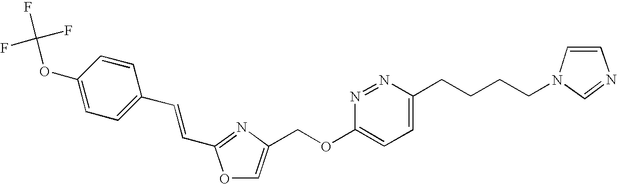

### a) 1-But-3-ynyl-1H-imidazole

To a solution of 1.54 g (23 mmol) imidazole in 50 ml dry tetrahydrofuran (THF) 0.54 g (23 mmol) sodium hydride were added in portions and the mixture was refluxed for one hour. After cooling down to room temperature 3.00 g (23 mmol) 4-bromo-but-1-yne were carefully added and the mixture was refluxed for three hours. After quenching with 5 ml water the mixture was evaporated to dryness and the residue was partitionated between diethyl ether and water. The layers were separated and the organic layer was dried over Na₂SO₄ and the solvent was removed in vacuo. Yield: 810 mg (10%) 1-but-3-ynyl-1H-imidazole as a colorless liquid.

MS: M=121.1 (ES+)

¹H-NMR (400 MHz, D₆-DMSO): δ=2.63 (td, 6.7 Hz, 2.7 Hz, 2H), 2.89 (t, 2.7 Hz, 1H), 4.08 (t, 6.7 Hz, 2H), 6.88 (s, 1H), 7.20 (s, 1H), 7.64 (s, 1H).

### b) 3-Chloro-6-(4-imidazol-1-yl-but-1-ynyl)-pyridazine

A mixture of 1.35 g (5.6 mmol) 3-chloro-6-iodo-pyridazine, 810 mg (6.7 mmol) 1-but-3-ynyl-1H-imidazole, 7.96 g (79 mmol) triethylamine, 107 mg (0.6 mmol) copper (I) iodide and 647 mg (0.6 mmol) tetrakis(triphenylphosphine) palladium (0) in 35 ml N,N-dimethylformamide (DMF) were stirred at room temperature overnight. After addition of 80 ml dichloromethane and 100 ml 0.5N HCl the phases were separated and the organic layer was dried over Na₂SO₄. Column chromatography (silica, ethyl acetate:methanol 3:1) returned 280 mg (22%) 3-chloro-6-(4-imidazol-1-yl-but-1-ynyl)-pyridazine as a white solid.

MS: M=233.0 (ES+)

¹H-NMR (400 MHz, D₆-DMSO): δ=3.12 (t, 6.6 Hz, 2H), 4.38 (t, 6.6 Hz, 2H), 7.32 (s, 1H), 7.60 (s, 1H), 7.83 (d, 8.8 Hz, 1H), 7.96 (d, 8.8 Hz, 1H), 8.49 (s, 1H).

### c) 3-Chloro-6-(4-imidazol-1-yl-butyl)-pyridazine

280 mg (3.2 mmol) 3-Chloro-6-(4-imidazol-1-yl-but-1-ynyl)-pyridazine in 50 ml MeOH were hydrogenated at room temperature in the presence of 120 mg PtO₂. The reaction mixture was filtered and concentrated in vacuo. Column chromatography (silica, ethyl acetate:methanol 3:1 to 1:1) returned 96 mg (34%) 3-chloro-6-(4-imidazol-1-yl-butyl)-pyridazine as a white solid.

MS: M=237.2 (ES+)

¹H-NMR (400 MHz, D₆-DMSO): δ=1.62 (quintet, 7.1 Hz, 2H), 1.74 (quintet, 6.9 Hz, 2H), 2.92 (t, 7.4 Hz, 2H), 3.99 (t, 6.7 Hz, 2H), 6.87 (s, 1H), 7.15 (s, 1H), 7.62 (s, 1H), 7.68 (d, 8.8 Hz, 1H), 7.83 (d, 8.8 Hz, 1H).

### d) 3-(4-Imidazol-1-yl-butyl)-6-{2-[(E)-2-(4-trifluoromethoxy-phenyl)-vinyl]-oxazol-4-ylmethoxy}-pyridazine

A mixture of 137 mg (0.5 mmol) {2-[2-(4-Trifluoromethoxy-phenyl)-vinyl]-oxazol-4-yl}-methanol, 94 mg (0.5 mmol) 3-Chloro-6-(4-imidazol-1-yl-butyl)-pyridazine and 50 mg (0.5 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 100 mg (42%) of the title compound as a light yellow solid.

MS: M=486.1 (APCI+)

¹H-NMR (400 MHz, D₆-DMSO): δ=1.62 (quintet, 7.7 Hz, 2H), 1.77 (quintet, 7.3 Hz, 2H), 2.85 (t, 7.6 Hz, 2H), 4.03 (t, 7.0 Hz, 2H), 5.41 (s, 2H), 6.98 (s, 1H), 7.19 (d, 9.1 Hz, 1H), 7.21 (d, 16.4 Hz, 1H), 7.23 (s, 1H), 7.40 (d, 8.5 Hz, 2H), 7.52 (d, 9.1 Hz, 1H), 7.58 (d, 16.4 Hz, 1H), 7.80 (s, 1H), 7.86 (d, 8.5 Hz, 2H), 8.23 (s, 1H).

### EXAMPLE 2

### 3-{2-[(E)-2-(4-Chloro-phenyl)-vinyl]-oxazol-4-ylmethoxy}-6-(4-[1,2,4]triazol-1-yl-butyl)-pyridazine

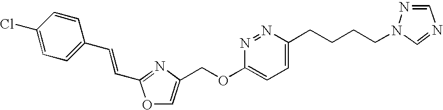

### a) 1-But-3-ynyl-1H-[1,2,4]triazole

A mixture of 10.0 g (145 mmol) 1,2,4-triazole, 6.21 g (155 mmol) sodium hydroxide and 17.17 g potassium iodide in 225 ml 2-methyl-butan-2-ol was refluxed for one hour. After cooling down to room temperature 15.33 g (103 mmol) methanesulfonic acid but-3-ynyl ester were added and the mixture was refluxed for three hours. The solvent was removed in vacuo and the residue was dissolved in water and extracted with ethyl acetate several times. The combined organic layers were dried over Na₂SO₄ and the solvent was removed in vacuo. Yield: 8.0 g (46%) 1-but-3-ynyl-1H-[1,2,4]triazole as a yellow liquid.

MS: M=122.1 (ES+)

¹H-NMR (400 MHz, D₆-DMSO): δ=2.71 (td, 6.7 Hz, 2.6 Hz, 2H), 2.87 (t, 2.6 Hz, 1H), 4.31 (t, 6.6 Hz, 2H), 7.98 (s, 1H), 8.53 (s, 1H).

### b) 3-Chloro-6-(4-[1,2,4]triazol-1-yl-but-1-ynyl)-pyridazine

A mixture of 2.00 g (8.3 mmol) 3-chloro-6-iodo-pyridazine, 1.21 g (10.0 mmol) 1-but-3-ynyl-1H-[1,2,4]triazole, 11.79 g (116 mmol) triethylamine, 175 mg (0.9 mmol) copper (I) iodide and 485 mg (0.4 mmol) tetrakis(triphenylphosphine) palladium (0) in 35 ml DMF were stirred at room temperature overnight. After addition of 80 ml dichloromethane and 100 ml 0.5N HCl the phases were separated and the organic layer was dried over Na₂SO₄. Column chromatography (silica, ethyl acetate:heptane 0:1 to 1:1) returned 1.30 g (67%) 3-chloro-6-(4-[1,2,4]triazol-1-yl-but-1-ynyl)-pyridazine as a white solid.

MS: M=234.0 (ES+)

¹H-NMR (400 MHz, D₆-DMSO): δ=3.11 (t, 6.4 Hz, 2H), 4.49 (t, 6.4 Hz, 2H), 7.78 (d, 8.9 Hz, 1H), 7.94 (d, 8.9 Hz, 1H), 8.03 (s, 1H), 8.65 (s, 1H).

### c) 3-Chloro-6-(4-[1,2,4]triazol-1-yl-butyl)-pyridazine

1.30 g (5.6 mmol) 3-Chloro-6-(4-[1,2,4]triazol-1-yl-but-1-ynyl)-pyridazine in 120 ml MeOH were hydrogenated at room temperature in the presence of 500 mg PtO₂. The reaction mixture was filtered and concentrated in vacuo. Column chromatography (silica, ethyl acetate:methanol 9:1) returned 290 mg (22%) 3-chloro-6-(4-[1,2,4]triazol-1-yl-butyl)-pyridazine as a white solid.

MS: M=238.2 (ES+)

¹H-NMR (400 MHz, D₆-DMSO): δ=1.65 (quintet, 7.8 Hz, 2H), 1.82 (quintet, 7.7 Hz, 2H), 2.93 (t, 7.6 Hz, 2H), 4.22 (t, 6.8 Hz, 2H), 7.68 (d, 8.9 Hz, 1H), 7.83 (d, 8.9 Hz, 1H), 7.95 (s, 1H), 8.51 (s, 1H).

### d) 3-{2-[(E)-2-(4-Chloro-phenyl)-vinyl]-oxazol-4-ylmethoxy}-6-(4-[1,2,4]triazol-1-yl-butyl)-pyridazine

A mixture of 107 mg (0.5 mmol) {2-[2-(4-chloro-phenyl)-vinyl]-oxazol-4-yl}-methanol, 94 mg (0.4 mmol) 3-Chloro-6-(4-[1,2,4]triazol-1-yl-butyl)-pyridazine and 47 mg (0.5 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 120 mg (60%) of the title compound as a light yellow solid melting at 165-167° C.

MS: M=437.3 (ES+)

¹H-NMR (400 MHz, D₆-DMSO): δ=1.62 (quintet, 7.6 Hz, 2H), 1.83 (quintet, 6.9 Hz, 2H), 2.85 (t, 7.6 Hz, 2H), 4.22 (t, 6.9 Hz, 2H), 5.40 (s, 2H), 7.19 (d, 8.6 Hz, 1H), 7.20 (d, 16.7 Hz, 1H), 7.47 (d, 8.6 Hz, 1H), 7.53 (d, 8.6 Hz, 2H), 7.54 (d, 16.7 Hz, 1H), 7.76 (d, 8.6 Hz, 2H), 7.95 (s, 1H), 8.22 (s, 1H), 8.51 (s, 1H).

### EXAMPLE 3

### 3-(4-[1,2,4]Triazol-1-yl-butyl)-6-{2-[(E)-2-(4-trifluoromethyl-phenyl)-vinyl]-oxazol-4-ylmethoxy}-pyridazine

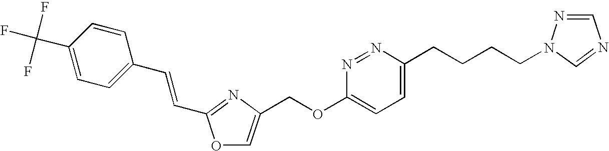

A mixture of 123 mg (0.5 mmol) {2-[2-(4-trifluoromethyl-phenyl)-vinyl]-oxazol-4-yl}-methanol, 90 mg (0.4 mmol) 3-Chloro-6-(4-[1,2,4]triazol-1-yl-butyl)-pyridazine and 47 mg (0.5 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 110 mg (51%) of the title compound as a white solid melting at 145-146° C.

MS: M=471.3 (ES+)

¹H-NMR (400 MHz, D₆-DMSO): δ=1.62 (quintet, 7.7 Hz, 2H), 1.83 (quintet, 7.3 Hz, 2H), 2.85 (t, 7.6 Hz, 2H), 4.22 (t, 6.9 Hz, 2H), 5.41 (s, 2H), 7.19 (d, 8.8 Hz, 1H), 7.34 (d, 16.4 Hz, 1H), 7.53 (d, 8.8 Hz, 1H), 7.63 (d, 16.4 Hz, 1H), 7.76 (d, 8.0 Hz, 2H), 7.94 (s, 1H), 7.95 (d, 8.0 Hz, 2H), 8.26 (s, 1H), 8.51 (s, 1H).

### EXAMPLE 4

### 3-{2-[(E)-2-(2-Fluoro-4-trifluoromethyl-phenyl)-vinyl]-oxazol-4-ylmethoxy}-6-(4-[1,2,4]triazol-1-yl-butyl)-pyridazine

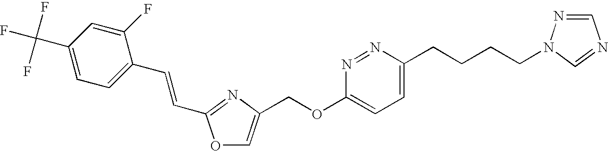

A mixture of 131 mg (0.5 mmol) {2-[2-(2-fluoro-4-trifluoromethyl-phenyl)-vinyl]-oxazol-4-yl}-methanol, 90 mg (0.4 mmol) 3-Chloro-6-(4-[1,2,4]triazol-1-yl-butyl)-pyridazine and 54 mg (0.6 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 42 mg (19%) of the title compound as a white solid melting at 141-143° C.

MS: M=489.3 (ES+)

¹H-NMR (400 MHz, D₆-DMSO): δ=1.62 (quintet, 7.5 Hz, 2H), 1.83 (quintet, 7.1 Hz, 2H), 2.85 (t, 7.6 Hz, 2H), 4.22 (t, 6.9 Hz, 2H), 5.42 (s, 2H), 7.19 (d, 9.1 Hz, 1H), 7.39 (d, 16.4 Hz, 1H), 7.53 (d, 9.1 Hz, 1H), 7.60 (d, 16.4 Hz, 1H), 7.64 (d, 8.6 Hz, 1H), 7.78 (d, 10.6 Hz, 1H), 7.96 (s, 1H), 8.15 (t, 7.7 Hz, 1H), 8.28 (s, 1H), 8.51 (s, 1H).

### EXAMPLE 5

### 2-(4-[1,2,4]Triazol-1-yl-butyl)-5-{2-[(E)-2-(4-trifluoromethyl-phenyl)-vinyl]-oxazol-4-ylmethoxy}-pyrazine

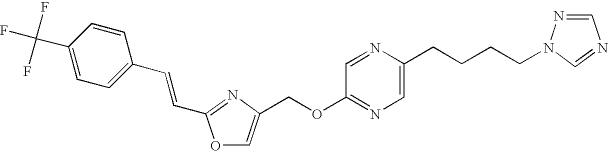

### a) 2-Bromo-5-(4-[1,2,4]triazol-1-yl-but-1-ynyl)-pyrazine

A mixture of 2.00 g (7.0 mmol) 2-bromo-5-iodo-pyrazine, 1.02 g (8.4 mmol) 1-but-3-ynyl-1H-[1,2,4]triazole, 9.95 g (98.3 mmol) triethylamine, 147 mg (0.8 mmol) copper (I) iodide and 404 mg (0.3 mmol) tetrakis(triphenylphosphine) palladium (0) in 30 ml DMF were stirred at room temperature overnight. After addition of 80 ml dichloromethane and 100 ml 0.5N HCl the phases were separated and the organic layer was dried over Na₂SO₄. Column chromatography (silica, ethyl acetate:heptane 0:1 to 1:1) returned 0.90 g (46%) 2-bromo-5-(4-[1,2,4]triazol-1-yl-but-1-ynyl)-pyrazine as a yellow solid.

MS: M=278.0 (ES+)

¹H-NMR (400 MHz, D₆-DMSO): δ=3.08 (t, 6.5 Hz, 2H), 4.47 (t, 6.5 Hz, 2H), 8.02 (s, 1H), 8.49 (s, 1H), 8.62 (s, 1H), 8.82 (s, 1H).

### b) 2-Bromo-5-(4-[1,2,4]triazol-1-yl-butyl)-pyrazine

900 mg (3.2 mmol) 2-Bromo-5-(4-[1,2,4]triazol-1-yl-but-1-ynyl)-pyrazine in 100 ml MeOH were hydrogenated at room temperature in the presence of 400 mg PtO₂. The reaction mixture was filtered and concentrated in vacuo. Column chromatography (silica, ethyl acetate:methanol 1:0 to 3:1) returned 300 mg (33%) 2-bromo-5-(4-[1,2,4]triazol-1-yl-butyl)-pyrazine as a light yellow solid.

MS: M=281.9 (ES+)

¹H-NMR (400 MHz, D₆-DMSO): δ=1.57-1.67 (m, 2H), 1.76-1.86 (m, 2H), 2.78 (t, 7.4 Hz, 2H), 4.20 (t, 6.8 Hz, 2H), 7.94 (s, 1H), 8.40 (d, 1.2 Hz, 1H), 8.50 (s, 1H), 8.75 (d, 1.2 Hz, 1H).

### c) 2-(4-[1,2,4]Triazol-1-yl-butyl)-5-{2-[(E)-2-(4-trifluoromethyl-phenyl)-vinyl]-oxazol-4-ylmethoxy}-pyrazine

A mixture of 105 mg (0.4 mmol) {2-[2-(4-trifluoromethyl-phenyl)-vinyl]-oxazol-4-yl}-methanol, 100 mg (0.4 mmol) 2-Bromo-5-(4-[1,2,4]triazol-1-yl-butyl)-pyrazine and 41 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 80 mg (48%) of the title compound as a light yellow solid melting at 147-148° C.

MS: M=471.2 (ES+)

¹H-NMR (400 MHz, D₆-DMSO): δ=1.65 (quintet, 7.4 Hz, 2H), 1.86 (quintet, 7.3 Hz, 2H), 2.77 (t, 7.4 Hz, 2H), 4.26 (t, 6.9 Hz, 2H), 5.35 (s, 2H), 7.38 (d, 16.4 Hz, 1H), 7.67 (d, 16.4 Hz, 1H), 7.81 (d, 8.3 Hz, 2H), 7.99 (s, 1H), 8.00 (d, 8.3 Hz, 2H), 8.16 (d, 1.2 Hz, 1H), 8.29 (s, 1H), 8.32 (d, 1.2 Hz, 1H), 8.56 (s, 1H).

### EXAMPLE 6

### 2-{2-[(E)-2-(4-Chloro-phenyl)-vinyl]-oxazol-4-ylmethoxy}-5-(4-[1,2,4]triazol-1-yl-butyl)-pyrazine

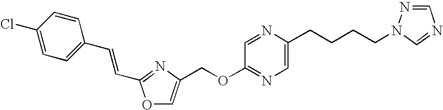

A mixture of 92 mg (0.4 mmol) {2-[2-(4-chloro-phenyl)-vinyl]-oxazol-4-yl}-methanol, 100 mg (0.4 mmol) 2-Bromo-5-(4-[1,2,4]triazol-1-yl-butyl)-pyrazine and 41 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 90 mg (58%) of the title compound as a white solid melting at 157-158° C.

MS: M=437.1 (ES+)

¹H-NMR (400 MHz, D₆-DMSO): δ=1.59 (quintet, 7.6 Hz, 2H), 1.80 (quintet, 7.3 Hz, 2H), 2.72 (t, 7.4 Hz, 2H), 4.20 (t, 6.9 Hz, 2H), 5.28 (s, 2H), 7.19 (d, 16.4 Hz, 1H), 7.47 (d, 8.4 Hz, 2H), 7.53 (d, 16.4 Hz, 1H), 7.75 (d, 8.4 Hz, 2H), 7.94 (s, 1H), 8.10 (d, 1.3 Hz, 1H), 8.20 (s, 1H), 8.26 (d, 1.3 Hz, 1H), 8.50 (s, 1H).

### EXAMPLE 7

### 2-(4-[1,2,4]Triazol-1-yl-butyl)-5-{2-[(E)-2-(4-trifluoromethoxy-phenyl)-vinyl]-oxazol-4-ylmethoxy}-pyrazine

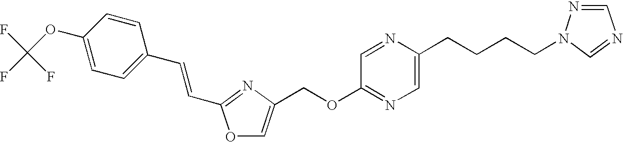

A mixture of 111 mg (0.4 mmol) {2-[2-(4-trifluoromethoxy-phenyl)-vinyl]-oxazol-4-yl}-methanol, 100 mg (0.4 mmol) 2-Bromo-5-(4-[1,2,4]triazol-1-yl-butyl)-pyrazine and 41 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 110 mg (64%) of the title compound as a white solid melting at 121-123° C.

MS: M=487.3 (ES+)

¹H-NMR (400 MHz, D₆-DMSO): δ=1.59 (quintet, 7.6 Hz, 2H), 1.80 (quintet, 7.4 Hz, 2H), 2.72 (t, 7.6 Hz, 2H), 4.21 (t, 6.9 Hz, 2H), 5.28 (s, 2H), 7.20 (d, 16.4 Hz, 1H), 7.40 (d, 8.4 Hz, 2H), 7.57 (d, 16.4 Hz, 1H), 7.86 (d, 8.4 Hz, 2H), 7.94 (s, 1H), 8.10 (d, 1.3 Hz, 1H), 8.21 (s, 1H), 8.26 (d, 1.3 Hz, 1H), 8.50 (s, 1H).

### EXAMPLE 8

### 5-(4-Pyrazol-1-yl-butyl)-2-{2-[(E)-2-(4-trifluoromethoxy-phenyl)-vinyl]-oxazol-4-ylmethoxy}-pyrimidine

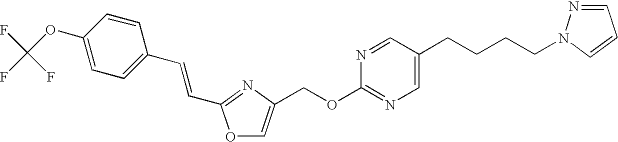

### a) 1-But-3-enyl-1H-pyrazole

A mixture of 5.0 g (7.3 mmol) pyrazole and 10.15 g (7.3 mmol) potassium carbonate in 100 ml butan-2-one were refluxed for one hour. After cooling down to room temperature 9.92 g (7.3 mmol) 4-bromo-but-2-ene were added and the mixture was heated to reflux overnight. After cooling down to room temperature the preticipate was filtered off and the filtrate was concentrated and purified by column chromatography (silica, ethyl acetate:heptane 1:1) to yield 1.04 g (12%) 1-but-3-enyl-1H-pyrazole as a yellow liquid.

MS: M=123.0 (ES+)

¹H-NMR (400 MHz, D₆-DMSO): δ=2.48-2.58 (m, 2H), 4.16 (t, 7.1 Hz, 2H), 4.96-5.08 (m, 2H), 5.74 (ddt, 17.4 Hz, 10.3 Hz, 6.8 Hz, 1H), 6.20 (t, 1.9 Hz, 1H), 7.41 (d, 1.7 Hz, 1H), 7.69 (d, 2.0 Hz, 1H)

### b) 2-Chloro-5-(4-pyrazol-1-yl-but-1-enyl)-pyrimidine

Under an atmosphere of nitrogen a mixture of 1.93 g (10 mmol) 5-bromo-2-chloropyrimidine, 1.04 g (8.5 mmol) 1-but-3-enyl-1H-pyrazole, 105 mg (0.4 mmol) triphenylphosphine, 2.73 g (27 mmol) triethylamine and 45 mg (0.2 mmol) palladium (II) acetate in 30 ml DMF were heated to 140° C. overnight. After cooling down to room temperature 60 ml 1N HCl were added and the aqueous layer was extracted three times with 80 ml ethyl acetate. The combined organic layers were dried over Na₂SO₄ and concentrated in vacuo. Column chromatography (silica, ethyl acetate:heptane 0:1 to 1:1) returned 540 mg (23%) 2-chloro-5-(4-pyrazol-1-yl-but-1-enyl)-pyrimidine as a yellow solid.

MS: M=235.1 (ES+)

¹H-NMR (400 MHz, D₆-DMSO): δ=2.72 (quartet, 6.9 Hz, 2H), 4.27 (t, 6.9 Hz, 2H), 6.21 (t, 2.0 Hz, 1H), 6.41 (d, 16.1 Hz, 1H), 6.57 (dt, 16.1 Hz, 6.9 Hz, 1H), 7.43 (d, 1.7 Hz, 1H), 7.73 (d, 2.0 Hz, 1H), 8.79 (s, 1H).

### c) 2-Chloro-5-(4-pyrazol-1-yl-butyl)-pyrimidine

540 mg (5.6 mmol) 2-chloro-5-(4-pyrazol-1-yl-but-1-enyl)-pyrimidine in 75 ml MeOH were hydrogenated at room temperature in the presence of 250 mg PtO₂. The reaction mixture was filtered and concentrated in vacuo. Column chromatography (silica, ethyl acetate) returned 280 mg (52%) 2-chloro-5-(4-pyrazol-1-yl-butyl)-pyrimidine as a light yellow.

MS: M=236.9 (ES+)

¹H-NMR (400 MHz, D₆-DMSO): δ=1.52 (quintet, 7.6 Hz, 2H), 1.77 (quintet, 7.3 Hz, 2H), 2.61 (t, 7.7 Hz, 2H), 4.12 (t, 6.9 Hz, 2H), 6.21 (t, 1.9 Hz, 1H), 7.41 (d, 1.8 Hz, 1H), 7.71 (d, 2.0 Hz, 1H), 8.64 (s, 2H).

### d) 5-(4-Pyrazol-1-yl-butyl)-2-{2-[(E)-2-(4-trifluoromethoxy-phenyl)-vinyl]-oxazol-4-ylmethoxy}-pyrimidine

A mixture of 120 mg (0.4 mmol) {2-[2-(4-trifluoromethoxy-phenyl)-vinyl]-oxazol-4-yl}-methanol, 90 mg (0.4 mmol) 2-Chloro-5-(4-pyrazol-1-yl-butyl)-pyrimidine and 49 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 110 mg (60%) of the title compound as a white solid melting at 143-145° C.

MS: M=486.2 (ES+)

¹H-NMR (400 MHz, D₆-DMSO): δ=1.50 (quintet, 7.3 Hz, 2H), 1.77 (quintet, 7.3 Hz, 2H), 2.54 (t, 7.6 Hz, 2H), 4.13 (t, 6.9 Hz, 2H), 5.28 (s, 2H), 6.21 (t, 2.0 Hz, 1H), 7.20 (d, 16.4 Hz, 1H), 7.40 (d, 8.4 Hz, 2H), 7.41 (d, 1.8 Hz, 1H), 7.57 (d, 16.4 Hz, 1H), 7.71 (d, 2.0 Hz, 1H), 7.86 (d, 8.4 Hz, 2H), 8.19 (s, 1H), 8.47 (s, 2H).

### EXAMPLE 9

### 5-(4-Pyrazol-1-yl-butyl)-2-{2-[(E)-2-(4-trifluoromethyl-phenyl)-vinyl]-oxazol-4-ylmethoxy}-pyrimidine

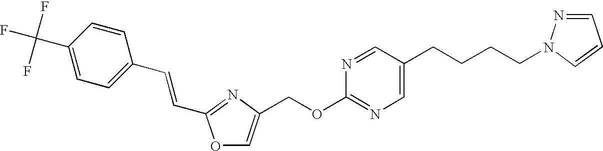

A mixture of 113 mg (0.4 mmol) {2-[2-(4-trifluoromethyl-phenyl)-vinyl]-oxazol-4-yl}-methanol, 90 mg (0.4 mmol) 2-Chloro-5-(4-pyrazol-1-yl-butyl)-pyrimidine and 44 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 80 mg (45%) of the title compound as a light yellow solid melting at 156-157° C.

MS: M=470.1 (ES+)

¹H-NMR (400 MHz, D₆-DMSO): δ=1.55 (quintet, 7.4 Hz, 2H), 1.82 (quintet, 7.1 Hz, 2H), 2.60 (t, 7.6 Hz, 2H), 4.18 (t, 6.9 Hz, 2H), 5.35 (s, 2H), 6.26 (t, 2.0 Hz, 1H), 7.39 (d, 16.4 Hz, 1H), 7.47 (d, 1.8 Hz, 1H), 7.67 (d, 16.4 Hz, 1H), 7.76 (d, 2.0 Hz, 1H), 7.81 (d, 8.1 Hz, 2H), 8.00 (d, 8.1 Hz, 2H), 8.28 (s, 1H), 8.52 (s, 2H).

### EXAMPLE 10

### 5-(4-Pyrazol-1-yl-butyl)-2-{2-[(E)-2-(4-chloro-phenyl)-vinyl]-oxazol-4-ylmethoxy}-pyrimidine

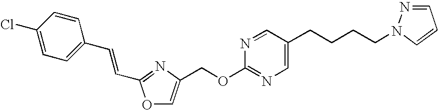

A mixture of 99 mg (0.4 mmol) {2-[2-(4-chloro-phenyl)-vinyl]-oxazol-4-yl}-methanol, 90 mg (0.4 mmol) 2-Chloro-5-(4-pyrazol-1-yl-butyl)-pyrimidine and 44 mg (0.4 mmol) sodium tert-butoxide in 5 ml THF were heated to 150° C. for 5 minutes in a microwave reactor. The reaction mixture was partitionated between ethyl acetate and saturated aqueous NH₄Cl. The phases were separated and the organic layer was dried over Na₂SO₄ and concentrated in vacuo. Preparative HPLC (RP18, methanol-water gradient) returned 110 mg (67%) of the title compound as a light yellow solid melting at 149-150° C.

MS: M=436.1 (ES+)

¹H-NMR (400 MHz, D₆-DMSO): δ=1.50 (quintet, 7.4 Hz, 2H), 1.77 (quintet, 7.3 Hz, 2H), 2.54 (t, 7.6 Hz, 2H), 4.13 (t, 6.9 Hz, 2H), 5.28 (s, 2H), 6.21 (t, 1.9 Hz, 1H), 7.19 (d, 16.4 Hz, 1H), 7.41 (d, 1.8 Hz, 1H), 7.47 (d, 8.4 Hz, 2H), 7.53 (d, 16.4 Hz, 1H), 7.71 (d, 2.0 Hz, 1H), 7.76 (d, 8.4 Hz, 2H), 8.18 (s, 1H), 8.47 (s, 2H).

1. A compound of formula I, 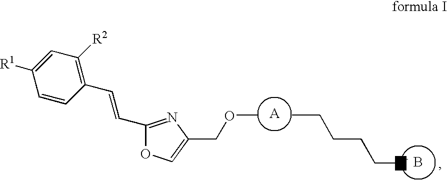
wherein
R¹ is selected from the group consisting of: halogenated alkyl, halogenated alkoxy and halogen;
R² is selected from the group consisting of: hydrogen and halogen;
ring A is selected from the group consisting of: 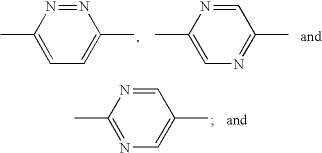
ring B is selected from the group consisting of: 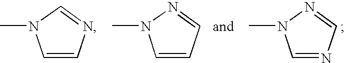
or a pharmaceutically acceptable salt thereof.

2. A compound according to claim 1, wherein R² is hydrogen.

3. A compound according to claim 1, wherein
R¹ is selected from the group consisting of: halogenated alkyl and halogenated alkoxy.

4. A compound according to claim 1, selected from the group consisting of:
3-(4-Imidazol-1-yl-butyl)-6-{2-[(E)-2-(4-trifluoromethoxy-phenyl)-vinyl]-oxazol-4-ylmethoxy}-pyridazine;
3-{2-[(E)-2-(4-Chloro-phenyl)-vinyl]-oxazol-4-ylmethoxy}-6-(4-[1,2,4]triazol-1-yl-butyl)-pyridazine;
3-(4-[1,2,4]Triazol-1-yl-butyl)-6-{2-[(E)-2-(4-trifluoromethyl-phenyl)-vinyl]-oxazol-4-ylmethoxy}-pyridazine;
3-{2-[(E)-2-(2-Fluoro-4-trifluoromethyl-phenyl)-vinyl]-oxazol-4-ylmethoxy}-6-(4-[1,2,4]triazol-1-yl-butyl)-pyridazine;
2-(4-[1,2,4]Triazol-1-yl-butyl)-5-{2-[(E)-2-(4-trifluoromethyl-phenyl)-vinyl]-oxazol-4-ylmethoxy}-pyrazine;
2-{2-[(E)-2-(4-Chloro-phenyl)-vinyl]-oxazol-4-ylmethoxy}-5-(4-[1,2,4]triazol-1-yl-butyl)-pyrazine;
2-(4-[1,2,4]Triazol-1-yl-butyl)-5-{2-[(E)-2-(4-trifluoromethoxy-phenyl)-vinyl]-oxazol-4-ylmethoxy}-pyrazine;
5-(4-Pyrazol-1-yl-butyl)-2-{2-[(E)-2-(4-trifluoromethoxy-phenyl)-vinyl]-oxazol-4-ylmethoxy}-pyrimidine;
5-(4-Pyrazol-1-yl-butyl)-2-{2-[(E)-2-(4-trifluoromethyl-phenyl)-vinyl]-oxazol-4-ylmethoxy}-pyrimidine; and
2-{2-[(E)-2-(4-Chloro-phenyl)-vinyl]-oxazol-4-ylmethoxy}-5-(4-pyrazol-1-yl-butyl)-pyrimidine.

5. A process for the manufacture of a compound according to formula I. 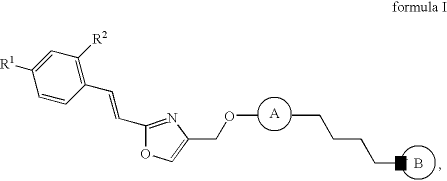
wherein
a compound of formula II 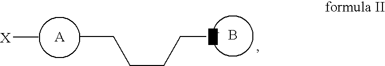
is reacted with a compound of formula III 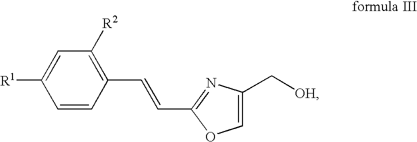
and wherein, in the above formulas,
R¹ is selected from the group consisting of: halogenated alkyl, halogenated alkoxy, and halogen;
R² is selected from the group consisting of: hydrogen and halogen;
ring A is selected from the group consisting of: 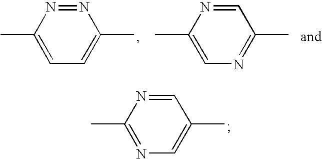
ring B is selected from the group consisting of: 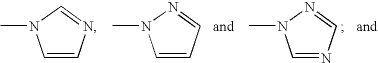
X is selected from the group consisting of: chlorine and bromine.

6. A pharmaceutical composition, containing a compound according to claim 1 and a pharmaceutically acceptable carrier.

7-8. (canceled)
